(19) United States
(12) Patent Application Publication
(10) Pub. No.: US 2024/0091050 A1
(43) Pub. Date: Mar. 21, 2024

(54) ADJUSTABLE ORAL APPLIANCE FOR TREATING SLEEP APNEA

(71) Applicant: SprintRay, Inc., Los Angeles, CA (US)

(72) Inventors: Mark Lorich, Los Angeles, CA (US); Amir Gordgi, Los Angeles, CA (US)

(21) Appl. No.: 18/370,141

(22) Filed: Sep. 19, 2023

Related U.S. Application Data
(60) Provisional application No. 63/408,051, filed on Sep. 19, 2022.

(51) Int. Cl. A61F 5/56

(52) U.S. Cl. CPC A61F 5/566

(57) ABSTRACT

An adjustable oral appliance for treating sleep apnea in a user. The oral appliance includes a first appliance frame and a second appliance frame. The first appliance frame includes a first tooth tray formed integrally with the first appliance frame to engage with user's upper teeth when worn, and the second appliance frame includes a second tooth tray formed integrally with the second appliance frame to engage with the user's lower teeth when worn. The first and second appliance frames include interlocking connectors to adjust the relative position of the second frame with respect to the first frame to set the user's upper and lower jaws in proper position, to move and raise the user's tongue, and to open the user's airway to avoid sleep apnea.

### FIELD OF THE INVENTION

The present invention generally relates to oral appliances, including oral appliances adapted to treat sleeping disorders.

### BACKGROUND

Sleep apnea is a sleep disorder characterized by abnormal pauses in breathing or instances of abnormally low breathing during sleep. Sleep apnea results from a partial-to-complete blockage of a subject's airway. Increased air speed through the airway causes an increase in dynamic pressure and a corresponding drop in static pressure. Sleep apnea may be either obstructive sleep apnea (OSA), central sleep apnea (CSA), or a combination of both.

Obstructive sleep apnea (OSA) is the most common sleep-related breathing disorder and is characterized by recurrent episodes of complete or partial obstruction of the upper airway leading to reduced or absent breathing during sleep. In OSA, the flow of air pauses or decreases during breathing while one is asleep, as the airway has become narrowed, blocked, or floppy.

OSA affects about 1 billion people between ages 30-69 globally, or roughly 1 in 10 people. Interruptions in sleep, often caused by snoring or airway obstruction, can cause weariness. Other mental and physical health conditions can also aggravate due to these complications. Treatments range from changing lifestyle habits, losing weight, avoiding alcohol, and surgeries such as palatal expansion and tonsillectomy. Furthermore, external devices such as CPAP or other suitable oral appliances also can be used for treating sleep apnea.

Traditionally, oral appliances are custom-fitted treatments created specifically for an individual patient by a clinician. Breathing patterns are monitored through sound or visually using CBCT imaging. Using this data, a clinician or technician attempts to determine an ideal jaw position for the patient. Moving the lower jaw forward (protrusive) while opening the bite (vertical) will open an airway, sometimes preventing sleep interruptions. Some patients may need to move backwards or close their bite to find the airway.

Some appliances are adjustable, which will increase the chance of success as the patient fine-tunes their sleep. Non-adjustable sleep appliances are less versatile and, as a result, require more input data for a successful design. However, Cone-beam computed tomography (CBCT) imaging is a costly barrier and will often increase patient's expenditure. Analog workflows for this appliance use a “George gauge” tool 100 as shown in FIG. 1 to lock a custom bite position for airway opening when fabricating models. The George gauge tool 100 is positioned inside the user's mouth as shown in FIG. 2. This position is easily found in the digital equivalence using dental CAD software. Sleep appliances, for example, thermoformed and milled custom sleep devices 300 and 400, typically, are made with thermoforming or milling machines as shown in FIG. 3 and FIG. 4, respectively. However, depending on a person's anatomy and malocclusion, these may not seat perfectly. The person's anatomy and malocclusion can cause even the 3D printed appliance to not seat properly. Special considerations for the design must be made, which also can be accommodated in the milled device. The thermoformed device has very limited methods to adapt in these situations, but they exist.

FIG. 3 shows the thermoformed device 300. This includes a pair of upper and lower splints, connected with metal fixtures and arms. These are rather tedious to assemble and can cause significant discomfort inside the mouth. Metal rubbing on cheeks causes irritation and sometimes minor cuts. Similar to orthodontic brackets, the method to alleviate this discomfort is placing a lump of dental wax on the metal as a temporary (nightly) cushion. This may not provide a good user experience. Again, as a thermoformed application, the scope of applicable mouths is limited in comparison to printed or milled.

FIG. 4 shows the milled device 400. The metal housings for the adjustable attachments are manually assembled after the milling process. This often leads to human errors and potentially affects the clinical function of the device. Also, as the attachment is adjusted, it moves further from the housing. This exposes metal and creates potentially hard to clean areas. The existence of this adjustment style, regardless of exposed metal or positioning of the attachment, may inevitably create a seam for microbes to invade.

Hence, there is a need for an improved sleep appliance design that remains hygienic for longer periods of time. There also is a need for more comfortable and less obtrusive oral appliances to treat sleep disorders.

### SUMMARY OF THE INVENTION

The present invention generally discloses an appliance for treating sleep apnea. Also, the present invention discloses an adjustable oral appliance configured to adjust the airway for treating sleep apnea while sleeping.

In some embodiments, the adjustable oral appliance/device is designed for treating sleep disorders in a user. The oral appliance is an innovative and intelligent sleep appliance for treating sleep disorders, such as sleep apnea. The oral appliance allows a user/patient to adjust their airway when sleeping. In some embodiments, the oral appliance comprises a first appliance or first appliance frame or upper appliance frame and a second appliance or second appliance frame or lower appliance frame.

In some embodiments, the first appliance frame is adapted for conforming to an upper jaw of the user. In some embodiments, the first appliance frame comprises a first tooth tray formed integrally with the first appliance frame. The first tooth tray includes a plurality of tooth indentations formed therein adapted to closely fit the teeth in the upper jaw of the user. In some embodiments, the first appliance frame further comprises a first connector. The first connector extends laterally at buccal surface of a right side and a left side towards anterior surface of the first appliance frame.

In some embodiments, the second appliance frame is adapted for conforming to a lower jaw of the user. In some embodiments, the second appliance frame comprises a second tooth tray formed integrally with the second appliance frame. The second tooth tray includes a plurality of tooth indentations formed therein adapted to closely fit the teeth in the lower jaw of the user. In some embodiments, the second appliance frame further comprises a second connector that extends vertically at buccal surface of a right side and a left side of the second appliance frame.

In some embodiments, the first appliance frame is attached to the second appliance via one or more connectors. In some embodiments, the one or more connectors are configured to maintain the first appliance frame and second appliance frame in locking position through yawning or other vertical movements. In some embodiments, the one or more connectors are hooks or anchors.

In some embodiments, the vertically extending second connector is attached to the laterally positioned first connector to maintain the first appliance frame and second appliance frame in locking position. In some embodiments, the second appliance frame is larger than the first appliance frame to keep the appliance in function during yawning, drinking, and other movements. In some embodiments, each appliance frame comprises a plurality of cuts, grooves, and connectors configured to maintain them in a locking position through yawning or other vertical movements.

In some embodiments, the oral appliance further comprises a concave surface configured to move and raise the tongue to lingual position to open the airway. In some embodiments, the oral appliance further utilizes a heat seat configured to adjust the dimensions or positions of the second appliance frame. In some embodiments, the oral appliance is an adjustable sleep appliance that provides automatic placement of the first appliance frame and second appliance frame. In some embodiments, the first appliance frame and second appliance frame are digitally altered in their width, which results in an adjustment to the bite's protrusion. To make vertical adjustments, variable thickness can be added to the occlusion of the oral appliance or edits can be made to attachments.

In some embodiments, the present invention utilizes a measurement tool. The measurement tool comprises at least one top notch at its upper section and a plurality of lower notches at its bottom section. The upper teeth will rest on the top notch, while the lower teeth will rest in one of the lower notches with labels. Adjusting the lower notch position will alter the protrusive opening of the bite. The position of attachments on the first appliance frame remains constant. The positions or dimensions of the second appliance frame is altered.

In some embodiments, the oral appliance functions as a dental deprogrammer. The deprogrammer resembles a retainer, night guard, or splint. The deprogrammer is provided with ramps or blocks. Minor adjustments to the bite position can be made over time, either through barely noticeable design changes, or significant additions like ramps or blocks. The deprogrammer is typically only worn on the upper arch. By locking the position of both arches, the oral appliance will function more efficiently. Also, contrary to typical ramped, blocked, or kois deprogrammers, the oral appliance will apply the jaw movement forces near the condyles of the jaw. In some embodiments, the oral appliance generates these forces in the anterior region of the mouth. By putting the forces closer to nature's intended location, the oral appliance functions superior than the existing devices.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

According to one aspect, one or more embodiments are provided below for an oral appliance for treating sleep apnea in a user, comprising a first appliance frame adapted to engage one or more upper teeth of a user when worn, the first appliance frame including an anterior side and a posterior side defining a first appliance longitudinal axis, a second appliance frame adapted to engage one or more lower teeth of the user when worn, a first connector coupled to the first appliance frame and a second connector coupled to the second appliance frame, wherein the first and second connectors are engageable with one another to restrict movement of the second appliance frame relative to the first appliance frame in a direction generally parallel to the first appliance longitudinal axis while enabling movement of the second appliance frame and of the first appliance frame in a direction generally perpendicular to the first appliance longitudinal axis.

In another embodiment, the first and second connectors are engageable with one another to restrict movement of the second appliance frame relative to the first appliance frame in a direction generally parallel to the first appliance longitudinal axis towards the posterior side of the first appliance frame.

In another embodiment, the first connector includes a gap adapted to receive at least a portion of the second connector.

In another embodiment, the second connector includes a gap adapted to receive at least a portion of the first connector.

In another embodiment, the second connector extends over at least a portion of the first frame.

In another embodiment, the first connector extends over at least a portion of the second frame.

The presently disclosed oral appliance and its method of manufacture and use is more fully described in the detailed description below.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 shows a conventional George gauge device.

FIG. 2 shows the George gauge device positioned inside a user's mouth in a typical use case.

FIG. 3 shows a conventional thermoformed custom sleep device.

FIG. 4 shows a conventional milled custom sleep device.

FIG. 5 shows a perspective view of an adjustable oral appliance according to some embodiments of the present invention.

FIGS. 6-8 show aspects of an adjustable oral appliance according to some embodiments of the present invention.

FIG. 9 shows aspects of a connector according to some embodiments of the present invention.

FIG. 10 shows placement of an adjustable oral appliance on a dental model according to some embodiments of the present invention.

FIGS. 11-12 show aspects of an adjustable oral appliance according to some embodiments of the present invention.

FIGS. 13-15 show a measurement tool according to different embodiments of the present invention.

FIGS. 16-18 show use of a measurement tool according to some embodiments of the present invention.

FIG. 19 shows a flowchart of a method for fabricating an adjustable oral appliance according to some embodiments of the present invention.

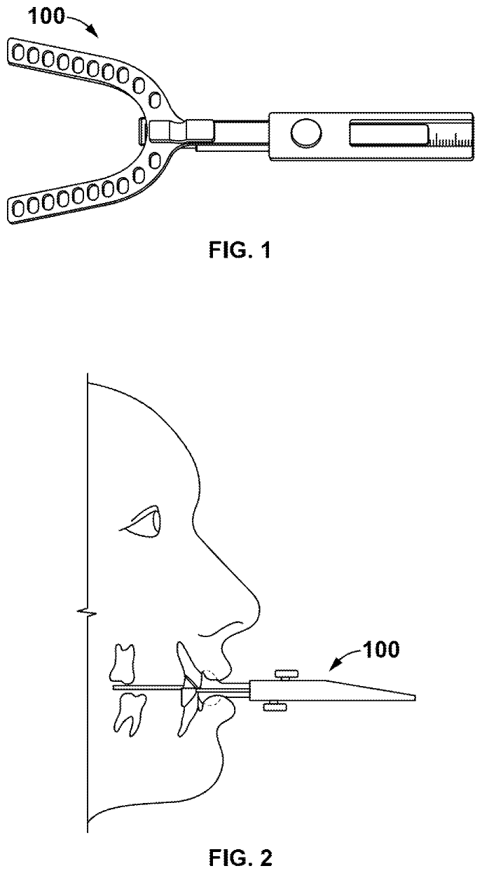

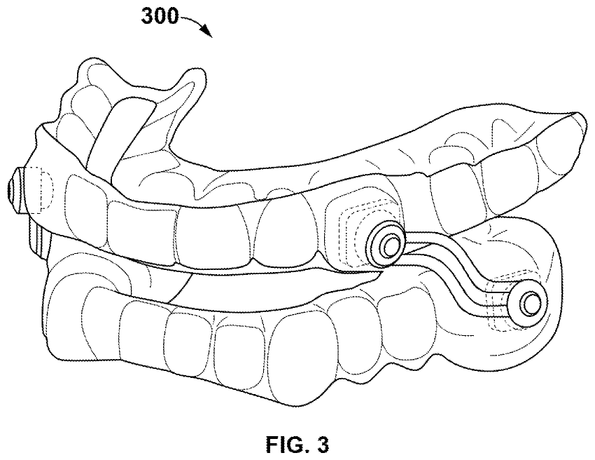

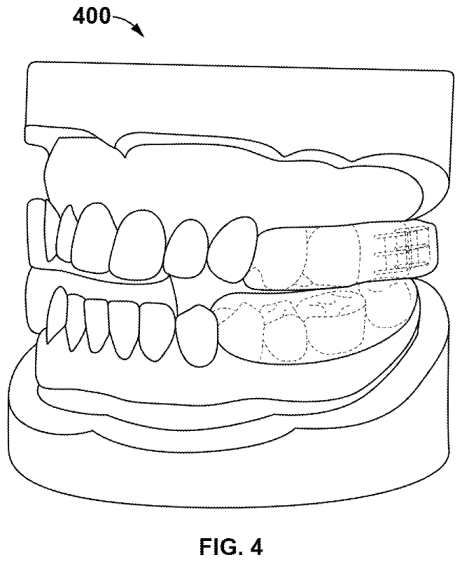

### DETAILED DESCRIPTION OF EMBODIMENTS

The present invention is best understood by reference to the detailed figures and description set forth herein.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

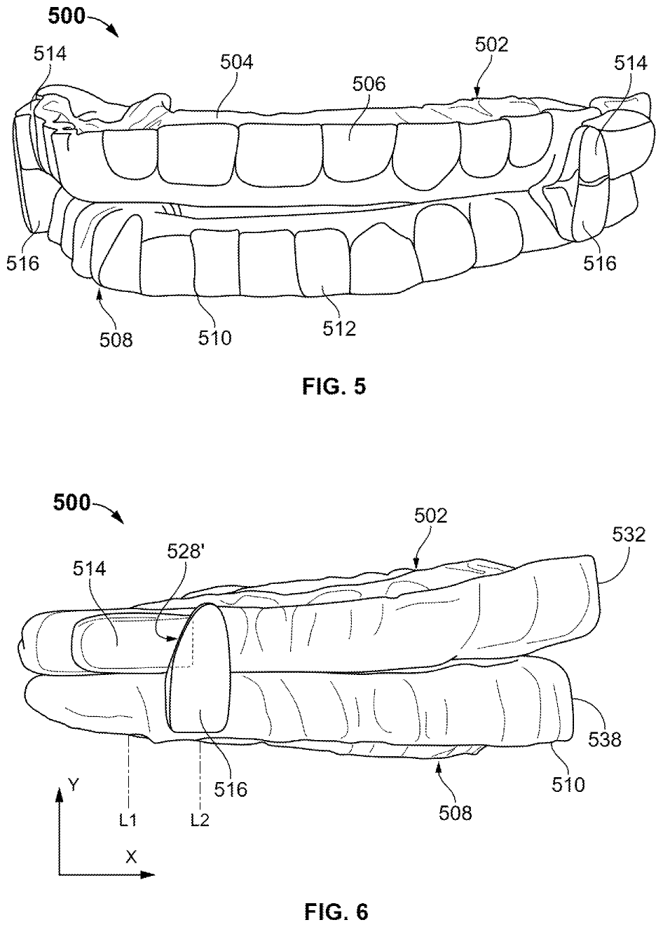

FIG. 5 shows a perspective view of an adjustable oral appliance/device or sleep appliance (hereinafter referred as an oral appliance) 500 for treating sleep disorder(s) in a user, according to some embodiments of the present invention. For example, the oral appliance 500 may include a sleep appliance for treating sleep disorders such as sleep apnea. The oral appliance 500 allows a user/patient to adjust the position of his/her airway and/or tongue when sleeping, thereby lessening the occurrences of sleep apnea.

In some embodiments, the oral appliance 500 may be additively manufactured, e.g., using rigid or flexible three-dimensional printer resin.

In some embodiments, the oral appliance 500 includes a first appliance frame 502 and a second appliance frame 508. In some embodiments, the appliance frames (502 and 508) may each include a splint appliance designed for the upper and lower jaws, respectively. In some embodiments, the appliances 502, 508 may be designed using dental CAD software.

In some embodiments, the first appliance frame 502 is designed to generally conform to an upper jaw and/or upper teeth of a user. The first appliance frame 502 includes a first tooth tray 504 formed integrally with the first appliance frame 502. In some embodiments, the first tooth tray 504 includes a plurality of tooth indentations 506 formed therein adapted to closely fit the teeth in the upper jaw of the user. In some embodiments, the second appliance frame 508 is designed to generally conform to the lower jaw and/or the lower teeth of the user. The second appliance frame 508 includes a second tooth tray 510 formed integrally with the second appliance frame 508. In some embodiments, the second tooth tray 510 includes a plurality of tooth indentations 512 formed therein adapted to closely fit the teeth in the lower jaw of the user.

As is known, sleep apnea may be reduced in a user by properly adjusting (e.g., properly opening) the user's airway during sleep. The user's tongue also may be properly positioned (e.g., raised) to help with the condition. This airway adjustment may be accomplished by properly setting the relative position of the user's upper and lower jaws and/or by setting the position of the user's tongue. For example, in some instances, a user's lower jaw may be moved and held in a slightly forward position with respect to the user's upper jaw to properly open the user's airway to treat sleep apnea.

In some embodiments, the first appliance frame 502 and the second appliance frame 508 are designed to hold the user's upper and lower jaws in a proper relative position to minimize the occurrence of sleep apnea. In some embodiments, this positioning may be achieved by corresponding connection mechanisms on the first and second frames 502, 508.

In some embodiments, the first appliance frame 502 includes one or more first connectors 514 and the second appliance frame 508 includes one or more second connectors 516. In some embodiments, the first connector(s) 514 on the first appliance frame 502 are designed to interlock with corresponding second connector(s) 516 on the second frame 508 and to hold the first and second frames 502, 508 (including the first and second tooth trays 504, 510) in a desired relative position with respect to one another during use.

In some embodiments, as shown in FIG. 5, a first pair of corresponding first and second connectors 514, 516 are located on the left buccal side of the first and second frames 502, 508, and a second pair of corresponding first and second connectors 514, 516 are located on the right buccal side of the first and second frames 502, 508. Other numbers and positions of the first and second connectors 514, 516 also may be used.

In a first example, in some embodiments, as shown in FIG. 6, a first connector 514 may be positioned at a first connector location L1 on the first frame 502 and a second connector 516 may be positioned at a second connector location L2 on the second frame 508. In this example, the first and second connector locations L1, L2 may be chosen to hold the lower appliance frame 508 at a particular position along the X-axis relative to the upper appliance frame 502. In this way, the user's lower teeth and lower jaw may be held in a position that properly opens the user's airway and/or positions the user's tongue to minimize the occurrence of sleep apnea.

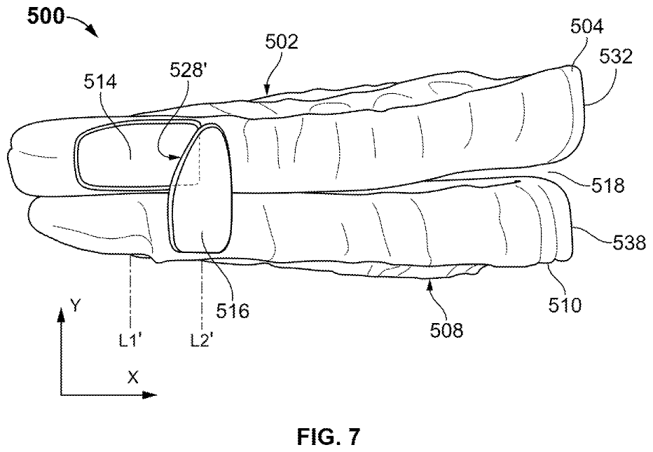

In a second example, in some embodiments, as shown in FIG. 7, the first connector 514 may be positioned at a first connector location L1′ on the first frame 502 and the second connector 516 may be positioned at a second connector location L2′ on the second frame 508. In this example, the first and second connector locations L1′, L2′ may be chosen to hold the lower appliance frame 508 at a slightly forward position compared to the position of the second frame 508 of FIG. 6. This increased forward position may further adjust the user's airway to minimize the occurrence of sleep apnea.

In some embodiments, the first and second connectors 514, 516 are designed and configured to interlock with one another and to maintain the first appliance frame 502 and second appliance frame 508 in a generally locked position while enabling the user to yawn, cough, and/or perform other generally vertical movements of his/her jaw without obstruction. Put another way, the first and second connectors 514, 516, when connected, may provide the first and second frames 502, 508 at least some degree of freedom in the Y-axis direction while generally maintaining the relative positions of the first and second frames 502, 508 in the X-axis direction. In this way, the first and second appliances 502, 508 may remain interconnected and in position within the user's mouth when the user yawns, coughs, or otherwise moves his/her jaw during sleep.

In some embodiments, the first and second connectors 514, 516 may include interlocking members such as hooks, anchors, wings, slots, grooves, recesses, cavities, openings, rods, tabs, gaps, other types of connectors, and any combinations thereof.

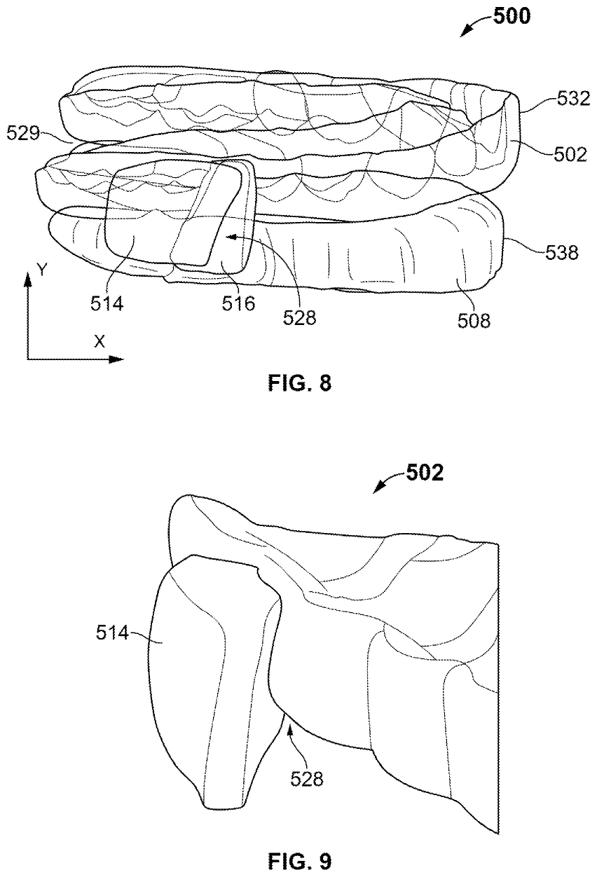

In some embodiments, as shown in FIGS. 8 and 9, the first connector 514 may be located at a buccal side of the first frame 502 and may extend laterally forward (towards the top frame's anterior surface 532) and slightly outward and away from the first frame 502 thereby forming a gap 528 between its forward end and the outer surface of the frame 502. A close-up view of the gap 528 is shown in FIG. 9. As shown in FIG. 8, the second connector 516 located at a corresponding buccal side of the second frame 508 may include a rear portion that is received into the gap 528 and releasably held therein (e.g., by pressure fit, friction, etc.). In this way, the first and second connectors 514, 516 may be interlocked along the X-axis but generally free to move to at least some degree along the Y-axis. In particular, because the gap 528 is located at the forward end of the first connector 514, backward movement of the second connector 516 is restricted by the body of the first connector 514. With the user's lower teeth held within the lower frame 508 (e.g., within the lower tooth tray 510) and the second connector 516 held within the gap 528, the lower frame 508 is held in place along the X-axis. Accordingly, the position of the gap 528 may generally set the position of the lower frame 508 (and therefore the position of the user's lower jaw) with respect to the upper frame 502 (and the user's upper jaw).

In some embodiments, while the gap 528 of FIG. 8 is formed between the first connector 514 and the first frame 502, it also is contemplated that the gap may be formed at the rear portion of the second connector 516 and the second frame 508 such that the first connector 514 may be received into the gap to hold the upper and lower frames 502, 508 in relative position. This is shown in FIGS. 6 and 7 and will be described in more detail in other sections.

In addition, in some embodiments, as shown in FIG. 8, the first connector 514, while connected to the first frame 502, may extend downward and overlap an outer portion of the second frame 508. Similarly, the second connector 516, while connected to the second frame 508, may extend upward and overlap an outer portion of the first frame 502. In this embodiment, the gap 528 may be formed between the first connector 514 and the upper frame 502 and between the first connector 514 and the lower frame 508, and the rear portion of the second connector 516 may be received into the gap 528 in either or both of these regions.

In other embodiments, as shown in FIGS. 6 and 7, the first connector 514 may be connected to the first frame 502 and may not necessarily extend downward to overlap the second frame 508, and the second connector 516, while connected to the second frame 508, may extend upward to overlap an outer portion of the upper frame 502. In this embodiment, the second connector 516 may form a gap 528′ between its rear portion and the outer surface of the upper frame 502 into which the forward portion of the first connector 514 may be received and held therein to lock the frames 502, 508 together.

In other embodiments, it also is contemplated that the first connector 514 may extend downward to overlay the second frame 508 thereby forming a gap between its forward portion and the outer surface of the second frame 508, and the second connector may not necessarily extend upwards to overlay the first frame 502. In this embodiment, the rear portion of the second connector 516 may be received into the gap and held therein to lock the frames 502, 508 together.

It is understood that either of the connectors 514, 516 may form gaps into which the corresponding connector 516, 514 may be received into, and that either or both of the connectors 514, 516 may extend beyond their respective frames 502, 508 to overlay the corresponding frame 508, 502. It also is contemplated that any combinations thereof of the described arrangements of the first and second connectors 514, 516 and/or of the first and second frames 502, 508 also are contemplated.

The first connector 514 and the second connector 516 are provided with more rounded designs, based on the feedback from the patients to avoid discomfort due to the sharp edges. In some embodiments, the oral appliance 500 further comprises a concave surface 529 configured to allow movement of the tongue to lingual position to open the airway.

During digital rendering, initially, the impressions or scan data, such as bite position, of the patient are acquired. The data is sent to the dental CAD software as input configured to create multiple splints (e.g., frames 502, 508) with offset attachments. The first and second appliance frames (502 and 508) are 3D-printed, post-processed, and delivered. The oral appliance 500 allows the user to adjust their airway by trying different appliance combinations as needed. Further, a clinician may take new measurements and collect feedback from the user/patient and apply changes to the design if the user further has sleep interruptions.

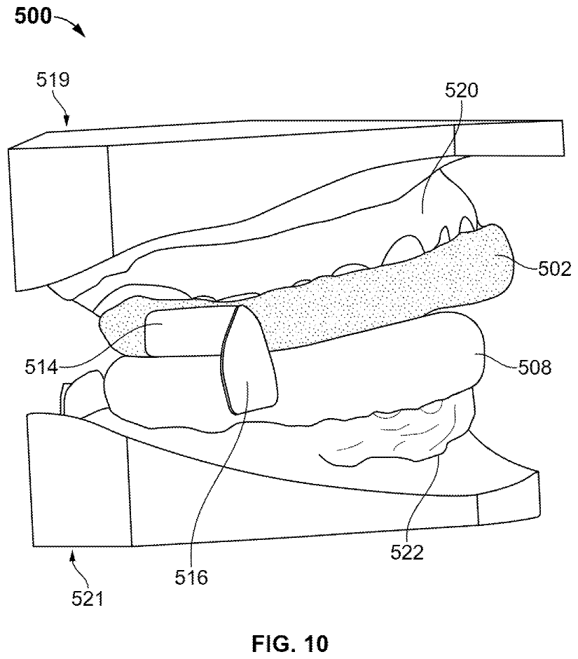

FIG. 10 illustrates a schematic view of a placement of the first and second frames 502, 508 onto a pair of 3D printed dental models (519, 521) including an upper arch 520 and a lower arch 522. The dental models 519, 521 may be formed using dental impressions or scan data taken from the user. In some embodiments, the dental models 519, 521 include a coordinate system used to verify the bite position of the user. The coordinate system is created during virtual articulation for the pair of 3D printed dental models (519, 521). The coordinate system provides a template for placing the first appliance frame 502 and second appliance frame 508 in a locked position. After fabrication, the first and second appliance frames (502 and 508) may be smoothed with a brush tool to prevent any discomfort to users/patients. In some embodiments, the protrusive and vertical movements of first and second appliance frames (502 and 508) are adjusted, thereby limiting the anterior contact to maintain the airway 518 or breathing pathway for the user.

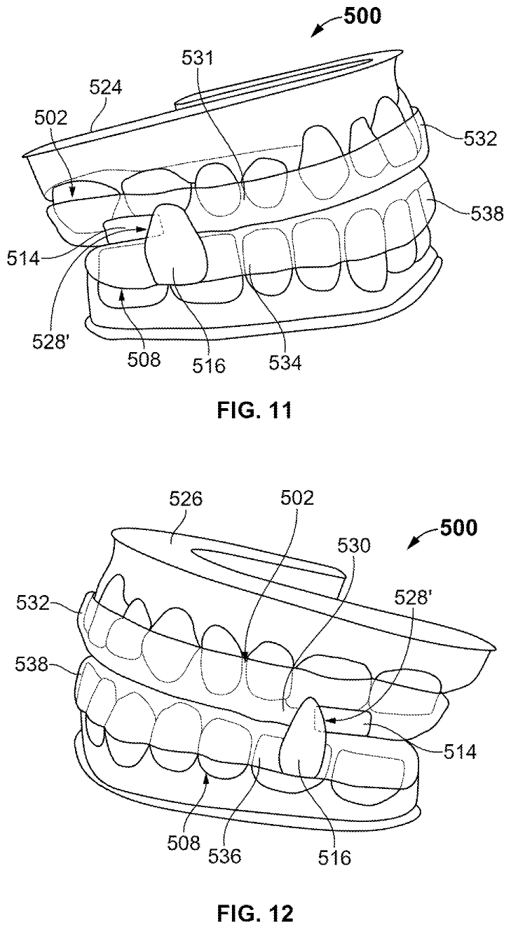

FIGS. 11-12 illustrate perspective views of a 3D-printed oral appliance 500 positioned over different dental models (524 and 526) configured to demonstrate the change in bite position, according to some embodiments of the present invention. In some embodiments, the first appliance frame 502 is attached to the second appliance frame 508 via one or more connectors 514, 516 as described in other sections. The connectors are configured to maintain the first appliance frame 502 and second appliance frame 508 in locking position through yawning or other vertical movements. In some embodiments, the one or more connectors may be hooks or anchors.

FIG. 11 shows the first connector 514 on the right buccal surface 531 of the first frame 502 extending laterally towards an anterior surface 532 of the first appliance frame 502. The second connector 516 on the right buccal surface 534 of the second frame 508 extends vertically upward to overlay a portion of the right buccal surface 531 of the first frame 502 and to form a gap 528′ that receives and secures a forward portion of the first connector 514.

Similarly, FIG. 12 shows the first connector 514 on the left buccal surface 530 of the first frame 502 extending laterally towards an anterior surface 532 of the first appliance frame 502. The second connector 516 on the left buccal surface 536 of the second frame 508 extends vertically upward to overlay a portion of the left buccal surface 536 of the first frame 502 and to form a gap 528′ that receives and secures a forward portion of the first connector 514.

In some embodiments, comparing the width of the second connector 516 in FIG. 11 to the width of the second connector 516 in FIG. 12, it is seen that the width of the second connector 516 in FIG. 11 is greater than the width of the second connector 516 in FIG. 12. For example, the second connector 516 shown in FIG. 11 overlays about half of a first tooth and a portion of a second tooth, while the second connector 516 shown in FIG. 12 overlays only a portion of a first tooth. In some embodiments, by increasing the width of the second connector 516 by extending the second connector 516 towards the rear of the second frame 508, the second frame 508 is moved forward thereby moving the user's lower jaw forward when worn. As such, in some embodiments, the dimensions of the first and/or second connectors 514, 516 (e.g., their widths) may be altered to adjust the relative position of the locked first and second frames 502, 508 as desired to alter the user's airway when used.

In some embodiments, each appliance frame (502 and 508) comprises a plurality of cuts, grooves, and connectors configured to maintain the frames 502, 508 in a locking position through yawning or other vertical movements.

In some embodiments, the oral appliance 500 provides automatic placement of first appliance frame 502 and second appliance frame 508. In some embodiments, the oral appliance 500 further utilizes a heat seat configured to adjust the dimensions or positions of the second appliance frame 508. The heat seat is designed to adjust the position of the appliance frames (502 and 508) and tooth indentations, creating a better fit to the patients' teeth and comfort around the gums. In some embodiments, the oral appliance 500 uses flexible resins with memory properties for frictionless patient adjustment. In some embodiments, the oral appliance 500 is an adjustable sleep appliance that provides automatic placement of the first appliance frame 502 and second appliance frame 508.

In some embodiments, the first connector 514 and the second connector 516 are digitally adjusted/altered in their width, which results in an adjustment to the bite's protrusion when the first and second frames 502, 508 are worn. To make vertical adjustments, variable thickness can be added to the occlusion of the oral appliance 500 or edits can be made to the first connector 514 and second connector 516. For example, if a patient has 6 slightly altered adjustable sleep appliances, they can mix-and-match to adjust their bite until they find a position that best prevents snoring.

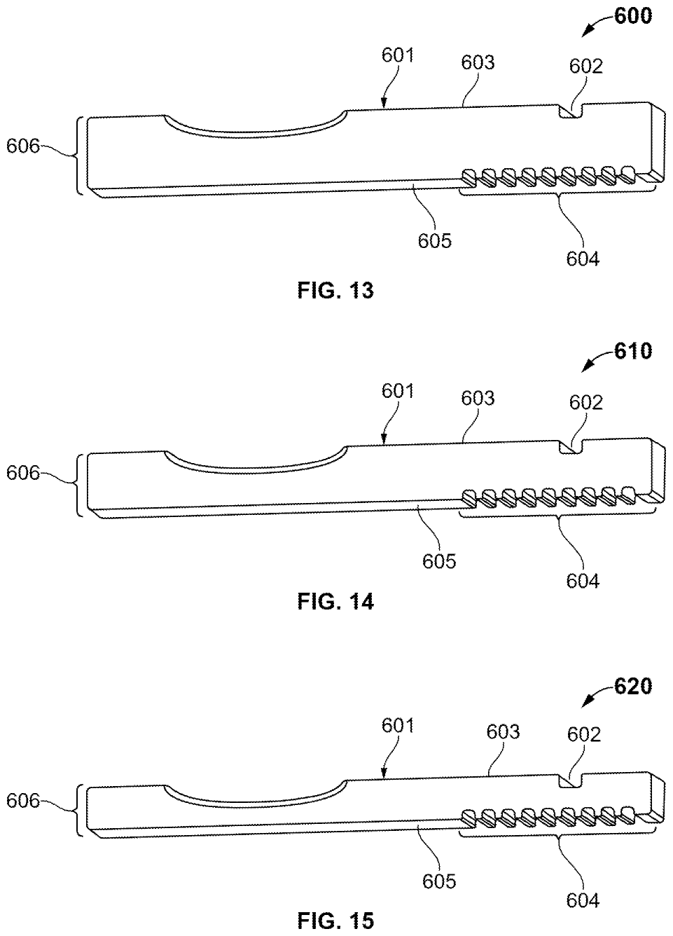

In some embodiments, the appliance 500 includes a measurement tool 600 that may be used to determine a proper alignment of the appliance's first and second frames 502, 508. In some embodiments, such a tool 600 is depicted in FIGS. 13-15. As shown, the tool 600 may include an elongate body member 601 including an upper surface 603 and a lower surface 605. In some embodiments, the upper surface includes an upper notch 602 and the lower surface 605 includes a plurality (e.g., 5-12) of sequential lower notches 604. The tool's body member 601 may include a height 606, and different versions of the tool 600 may include different body member heights 606. For example, a first tool 600 may include a body member height 606 of about 8 mm (e.g., in FIG. 13) and a second tool 610 may include a body member height 606 of about 6 mm (e.g., in FIG. 14), and a third tool 620 may include a body member height 606 of about 4 mm (e.g., in FIG. 15). It is understood that the height 606 of the tool 600 may be chosen to be any value as needed for proper use of the tool, e.g., between 2 mm to about 20 mm. As will be described in other sections, the height 606 of the tool 600 may determine the vertical opening of the user's bite during use of the tool 600. In addition, other tools 600 of other heights 606 also may be used.

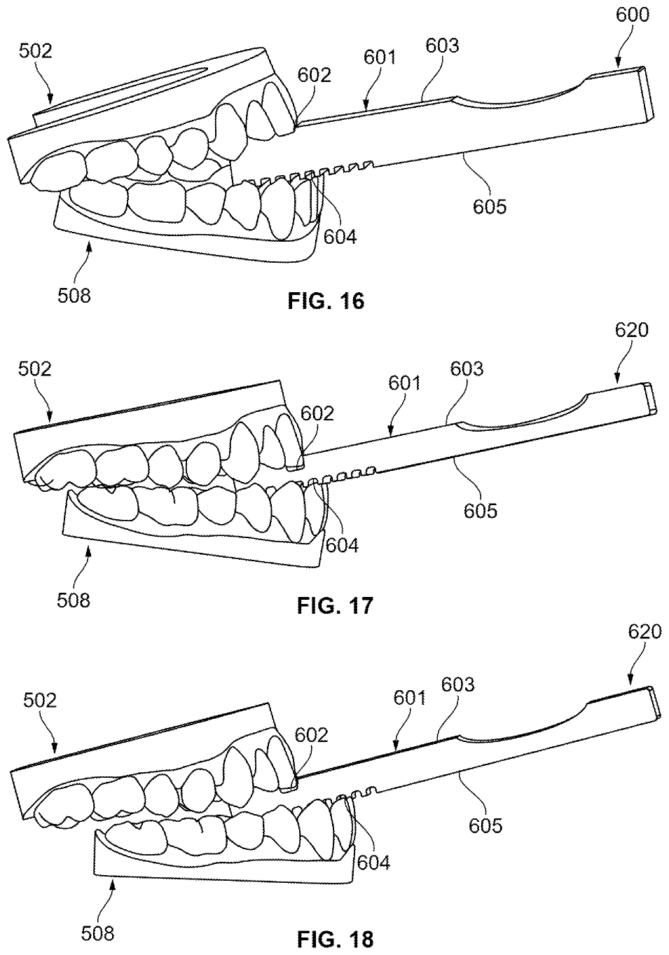

In some embodiments, as shown in FIGS. 16-18, the tool 600, 610, 620 may be used to set and measure various relative positions of the first and second frames 502, 508 worn by the user. In some embodiments, the tool 600 may be placed into the user's mouth such that the user's upper teeth within the anterior side 532 of the first frame 502 rest within the tool's upper notch 602. The user's bottom teeth within the anterior side 538 of the lower frame 508 may then be placed into one of the tool's lower notches 604. Depending on which lower notch 604 the lower teeth are placed into, the relative position of the lower frame 508 with respect to the upper frame 502 may be set. That is, while holding the position of the upper frame 502 constant using the single upper notch 602, the user's lower teeth may be placed into various lower slots 604 to adjust the protrusive opening of the user's bite.

In some embodiments, a tool 600, 610, 620 with a specific height 606 may be chosen and inserted into the user's mouth. Then, the with the user's upper teeth held within the tool's upper notch 602, the user's lower teeth may be systematically placed into one or more of the lower notches 604 and the user may breathe and attempt to intentionally snore. A clinician working with the user may systematically change which lower slot 604 is used until a setting is determined that may properly adjust the user's airway and/or tongue to reduce the occurrence of sleep apnea.

In some embodiments, the lower notches 604 each include a unique label so that the notches may be identified during use. When the clinician identifies a proper tool height 606 and a proper lower notch 604 setting, he/she may note which lower notch 604 was used. This may provide the clinician with relative spatial information (e.g., measurements) regarding the user's jaws and the respective first and second frames 502, 508. This information may then be used during fabrication of the first and second frames 502, 508, and specifically to set the proper positions of the first and second connectors 514, 516 on the first and second frames 502, 508 to result in the spacing of the frames 502, 508 during use of the appliance 500 as determined by use of the tool 600. This procedure may reduce burden on the manufacturing workflow.

In some embodiments, an ideal position of the first and second frames 502, 508 may be communicated to a CAD designer, who may use these settings as a template for standardized positioning of the sleep appliance 500 and its respective connectors 514, 516. Before of this implementation, multiple options for optimum positioning of the attachments also are provided. That is, a series of appliances 500 including a series of first frames 502 and a series of corresponding second frames 508 with various connector 514, 516 positions may be fabricated for use by the user. The user may mix and match the various first and second frames 502, 508 with different connector 514, 516 positions to determine which pair(s) may work best. As such, use of the tools 600, 610, 620 may beneficially streamline the optimization of the appliance 500.

In some embodiments, the measurement tools (600, 610, 620) can also be replaced or supplemented by jaw motion capture scans, Cone-beam computed tomography (CBCT) data for airway opening, or a custom bite scan. The ideal relative position of the first and second frames 502, 508 also can be determined using AI/automation.

In some embodiments, the oral appliance 500 functions as a dental deprogrammer. The deprogrammer resembles a retainer, night guard, or splint. The deprogrammer is provided with ramps or blocks. Minor adjustments to the bite position can be made over time, either through barely noticeable design changes, or significant additions like ramps or blocks. Traditional deprogrammers are typically only worn on the upper arch. However, by using both an upper frame 502 and a lower frame 508 in combination, and by thereby locking the position of the user's both arches, the oral appliance 500 will function more efficiently. Also, contrary to typical ramped, blocked, or kois deprogrammers, the oral appliance 500 may apply the jaw movement forces near the condyles of the jaw. In some embodiments, the oral appliance 500 generates these forces in the anterior region of the mouth. By putting the forces closer to nature's intended location, the oral appliance 500 functions superior to the existing devices.

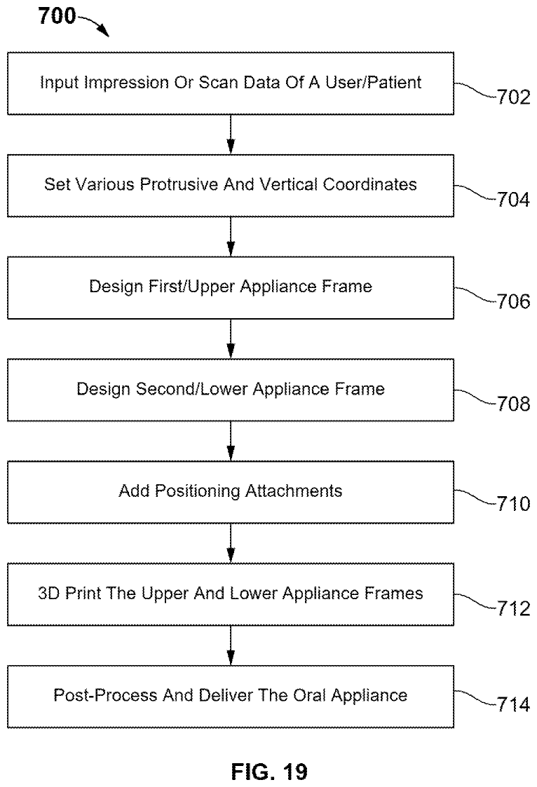

FIG. 19 illustrates a flowchart 700 of a design method for 3D-printing the oral appliance 500, according to some embodiments of the present invention. The design method allows a designer to simulate multiple jaw positions based on the patient's anatomy. After the designer establishes a few ideal setups, he/she may manually add attachments (e.g., the first and second connectors 514, 516) to the oral appliance 500. The angulation of the first appliance frame or upper arch attachment 502 affects the vertical movements, and the extrusion of the second appliance frame or lower arch attachment 508 affects the protrusive movements. This workflow enables the treatment without requiring the user of extra hardware, such as a CBCT or George gauge.

In some embodiments, the method comprises the following steps. At step 702, a file having a plurality of data including impressions and scans of a user/patient is transferred as input to a dental CAD software.

At step 704, various protrusive and vertical coordinates are set by the user. At step 706, the first/upper appliance frame 502 is designed using impressions collected from the user. At step 708, the second/lower appliance frame 508 is designed using impressions collected from the user. At step 710, positioning attachments such as first connector 514 and second connector 516 are added to the first and second frames 502, 508. At step 712, the design file is transferred to a 3D printer as input. In some embodiments, the 3D printer includes a slicing software or cloud interface. In some embodiments, the design file is oriented, supported, and responsively added to the layout and queued with similar models using the cloud software. In some embodiments, the designer may set up the design file on desktop software. The 3D printer may then print the first appliance frame 502 and the second appliance frame 508. After printing, the frames (502 and 508) may be washed and cured.

At step 714, the 3D-printed appliance frames (502 and 508) are post-processed, and the oral appliance is delivered. In some embodiments, after curing, additional post-processing is performed using ultrasonic cleaning or warm soapy water. Optional polishing is possible with a lathe, wheel, and series of pumice, steam, and polishing compounds. This process obfuscates the layer lines from 3D-printing and makes the oral appliance 500 nearly indistinguishable from a thermoformed equivalent. The polishing process helps the patient with aftercare, as it is easier to clean.

Optionally, in some embodiments, a “heat seat” delivery method may be used. The heat seat method involves placing the oral appliance 500 in warm water having the temperature ranges between 95-115° F. for about 10-20 seconds to soften the first and second frames 502, 508 such that they are malleable. The malleable frames 502, 508 may then be placed into position in the user's mouth such that when the frames harden 502, 508, they are properly adapted to the patient's teeth.

Advantageously, the oral appliance of the present invention allows a patient to adjust their airway when sleeping. The design of the oral appliance enables the treatment without extra hardware, such as the CBCT or George gauge. The oral appliance could be easily designed by any office with the capability of 3D-printing a typical dental splint.

Preferred embodiments of this invention are described herein, including the best mode known to the inventors for carrying out the invention. It should be understood that the illustrated embodiments are exemplary only and should not be taken as limiting the scope of the invention.

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

What is claimed is:

1. An oral appliance for treating sleep apnea in a user, comprising:
a first appliance frame adapted to engage one or more upper teeth of a user when worn, the first appliance frame including an anterior side and a posterior side defining a first appliance longitudinal axis;
a second appliance frame adapted to engage one or more lower teeth of the user when worn;
a first connector coupled to the first appliance frame and a second connector coupled to the second appliance frame;
wherein the first and second connectors are engageable with one another to restrict movement of the second appliance frame relative to the first appliance frame in a direction generally parallel to the first appliance longitudinal axis while enabling movement of the second appliance frame and of the first appliance frame in a direction generally perpendicular to the first appliance longitudinal axis.

2. The oral appliance of claim 1 wherein the first and second connectors are engageable with one another to restrict movement of the second appliance frame relative to the first appliance frame in a direction generally parallel to the first appliance longitudinal axis towards the posterior side of the first appliance frame.

3. The oral appliance of claim 1 wherein the first connector includes a gap adapted to receive at least a portion of the second connector.

4. The oral appliance of claim 1 wherein the second connector includes a gap adapted to receive at least a portion of the first connector.

5. The oral appliance of claim 1 wherein the second connector extends over at least a portion of the first frame.

6. The oral appliance of claim 1 wherein the first connector extends over at least a portion of the second frame.

7. The oral appliance of claim 1, further comprising a concave surface configured to move and raise a tongue of the user to a lingual position to open an airway of the user.
